(19) United States
(12) Patent Application Publication
(10) Pub. No.: US 2005/0228355 A1
(43) Pub. Date: Oct. 13, 2005

(54) Feminine protection kit

(76) Inventor: Carla Renae Briggs, Indianapolis, IN (US)

Correspondence Address: THE INVENTORS NETWORK, INC., 332 ACADEMY STREET, CARNEGIE, PA, 15106, US

(21) Appl. No.: 10/822,204

(22) Filed: Apr. 12, 2004

(51) Int. Cl. A61F 13/15

(52) U.S. Cl. 604/385.06; 206/438; 206/363

(57) ABSTRACT

A feminine hygiene protection and freshening kit includes a lightweight, waterproof packaging containing therein a feminine pad, tampon or napkin and a feminine wipe attached thereto. The feminine wipe is enclosed within a wrapper for sanitary purposes, and after the feminine wipe is used both for cleaning and freshening, the feminine wipe and the used pad, tampon or napkin are placed in the packaging for easy and sanitary disposal.

### FIELD OF THE INVENTION

The present invention pertains to hygiene kits, and more particularly pertains to a easily transportable, unobtrusive, lightweight feminine hygiene protection kit.

### BACKGROUND OF THE INVENTION

The maintenance of personal hygiene is a vital concern for females, and the concern is amplified at the onset of menstruation and throughout the menstrual cycle. Such onsets occur without warning and often at the most inconvenient and unexpected times. Thus, it is generally impossible to be prepared for every contingency for when this may occur, and the individual, especially an active, on-the-go female, will not wish to restrict her activities in order to simply await the onset of menstruation. Therefore, it is desirable to have some type of feminine hygiene item or product readily available for wiping and cleaning purposes and also a feminine protection item in the form of a napkin, pad, or tampon for providing continued protection thereafter. Such feminine hygiene items or products should be lightweight and unobtrusive so they can be carried in one's pocket, purse, or handbag so as to not impede the individual's diverse activities; and they should also be simple and easy to employ and capable of simple and efficient sanitary disposal.

Thus, the prior art discloses a number of devices and products for maintaining and enhancing female personal hygiene.

For example, the Anderson patent (U.S. Pat. No. 4,896,768) that discloses a presaturated wipe product that includes an irradiated pad of dilute glutaraldehyde attached to a layer of packaging material and which maintains its disinfectant activity long term due to the prior irradiation.

The Finkel et al. patent (U.S. Pat. No. 4,702,378) discloses a baby change kit that includes a dry towel, a wet towel, baby powder and soap contained within a foldable plastic film container.

The Gerry patent (U.S. Pat. No. 4,901,851) discloses a feminine cleaning package that includes a pair of sponges sealed within respective waterproof packets both of which are disposed within a waterproof package.

The Nieves patent (U.S. Pat. No. 5,261,531) discloses a feminine hygiene package that includes a dry wipe, a wet wipe and a sanitary napkin stored within an enclosure.

The Reed patent (U.S. Pat. No. 5,740,554) discloses a mitten device for removing feminine hygiene products and which can be inverted after removal of the product for disposal therewith.

The James patent (U.S. Pat. No. 6,059,100) discloses a feminine hygiene disposal system that includes a main container storing therein a plurality of separable disposable containers.

### SUMMARY OF THE INVENTION

A lightweight, easily portable feminine hygiene protection kit includes a packaging having a cavity or chamber for storing therein a feminine protection item and a feminine wipe. The feminine wipe is enclosed in a wrapping for maintaining the sanitary qualities of both the wipe and the protection item, and the wipe can be scented to enhance the cleansing and freshening process, especially during female menstrual cycle times. After the wipe has been used and the used feminine protection item has been removed, both the used wipe and the used protection item can be placed in the packaging for safe and sanitary disposal in, for example, a refuse container.

It is an objective of the present invention to provide a feminine protection kit that provides females with a fresh physical sense after each change of a feminine napkin, pad or tampon.

It is another objective of the present invention to provide a feminine protection kit that is convenient for active, on-the-go women.

It is yet another objective of the present invention to provide a feminine protection kit that eliminates the need for a female to use a wet tissue for personal cleansing purposes and for carrying more than one feminine hygiene product.

It is still another objective of the present invention to provide a feminine protection kit that is easily portable and can be carried in a pocket, purse or handbag.

Yet still another objective of the present invention is to provide a feminine protection kit that is inexpensive and that can be easily and safely disposed.

A yet still further objective of the present invention is to provide a feminine protection kit that saves storage space and gives enhanced comfort to females at those critical times each month.

Still a yet further objective of the present invention is to provide a feminine protection kit wherein the packaging of the kit can be used as a disposal container for the napkin, pad, or tampon and the feminine wipe.

These and other objects, features and advantages will become apparent to those skilled in the art upon a perusal of the following detailed description read in conjunction with the accompanying drawing figures.

### BRIEF DESCRIPTION OF THE DRAWINGS

FIG. 1 is a perspective view of the feminine protection kit of the present invention illustrating a square-shaped container being opened to remove a pad-shaped feminine hygiene product and a feminine wipe attached thereto;

FIG. 2 is a sectioned elevational view of the feminine hygiene kit shown in FIG. 1;

FIG. 3 is a perspective view of a feminine protection kit illustrating a cylindrical-shaped feminine hygiene product encircled by a feminine wipe;

FIG. 4 is a sectioned elevational view of the feminine hygiene kit shown in FIG. 3; and

FIG. 5 is a perspective view illustrating the disposal of the feminine hygiene kit first shown in FIG. 1 wherein the feminine hygiene product and the feminine wipe are placed in the package for disposal in the rubbish container.

### DETAILED DESCRIPTION OF THE PREFERRED EMBODIMENT

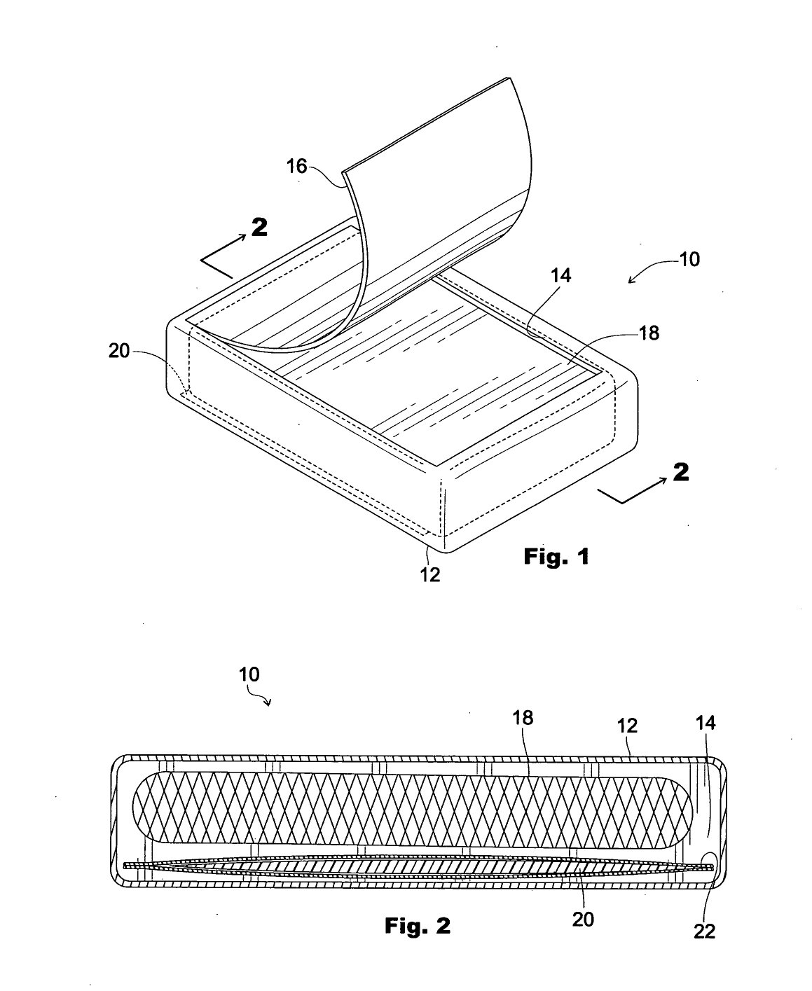

Illustrated in FIGS. 1-5 are several embodiments of a portable feminine hygiene protection kit that can be carried by a female for easy use and safe and sanitary disposal. Shown in FIGS. 1 and 2 is a feminine hygiene protection kit 10 that includes a lightweight, waterproof container or packaging 12 that includes a holding cavity 14 and a lift up or peel off lid 16 for accessing the contents held within the cavity 14. The primary components stored within the packaging 12 are a feminine protection item 18 such as a pad, tampon or napkin and a moistened feminine wipe 20 to provide for adequate cleansing after removal of the used tampon. The feminine wipe 20 is available in at least three different scents that permeate the wipe 20: regular, lightly scented, or moistened. The feminine wipe 20 is enclosed in an easy-to-open wrapper 22 for preserving the sanitary, scented or medicated qualities of the feminine wipe 20. To improve its quick and efficient removal from the packaging 12, the feminine wipe 20 can be attached to the feminine protection item 18 so that both components can be quickly removed together from the packaging 12 thus avoiding the problem of having them drop on the floor and separate from each other.

In using the feminine hygiene protection kit 10 of FIG. 1 and 2, the female would open the lid 16 and remove the protection item 18 and the attached feminine wipe 20 that could be a pad, tampon or napkin. After cleansing herself with the feminine wipe 20 and then changing to the new feminine protection item 18, the female user would then place the feminine wipe 20 and the used protection item 18 (the tampon, pad or napkin) in the cavity 14 of the packaging 12. The wipe 20 and the used tampon, for example, would then be safely and sanitarily disposed of along with the packaging 12 in a refuse container 24, as shown in FIG. 5. The female user is thus provided with a convenient, easily storable, fresh, clean alternative to employing dry toilet paper, and the female user would be given a renewed feeling of freshness after changing her protection.

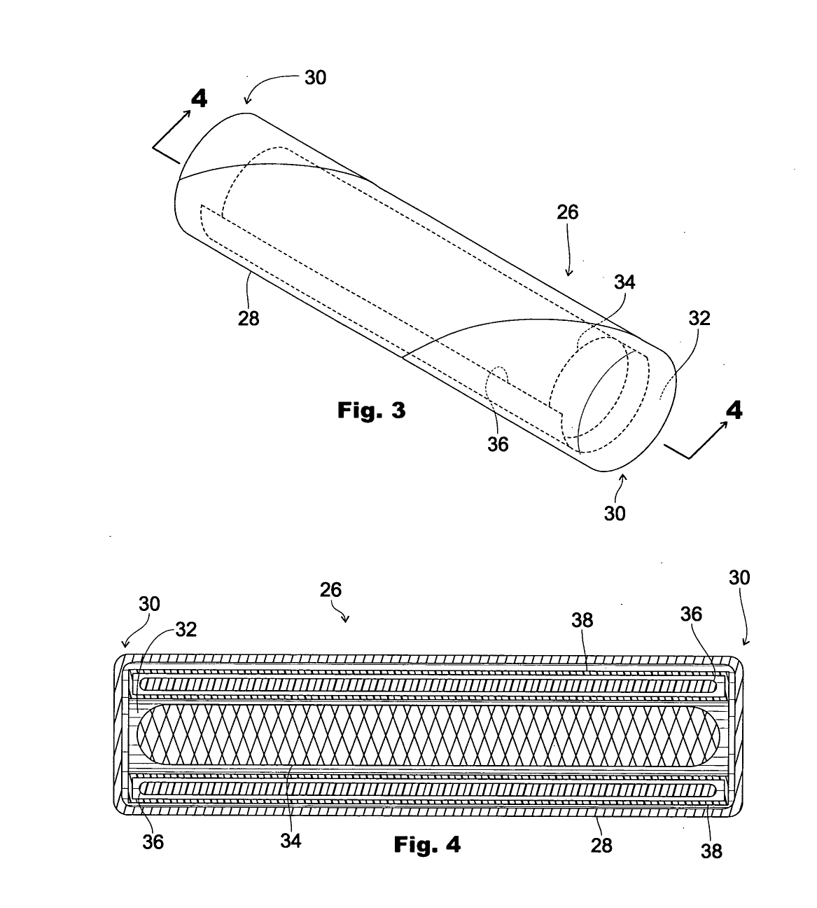

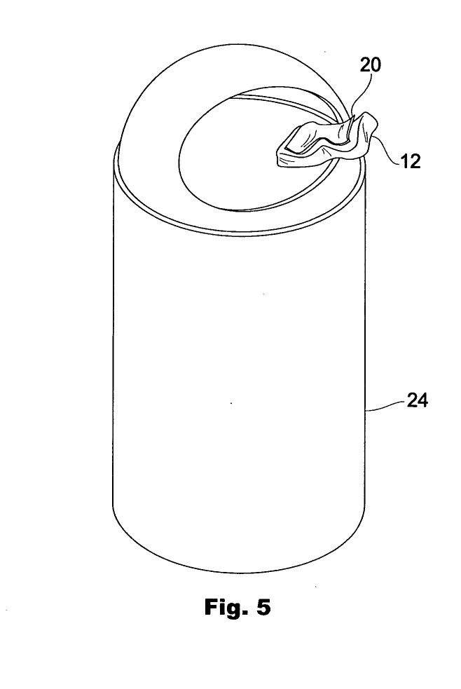

FIGS. 3 and 4 illustrate another embodiment for the feminine hygiene protection kit that can be unobtrusively carried by the female for easy and quick use and disposal. The feminine hygiene protection kit 26 of FIGS. 3 and 4 includes a cylindrical container or packaging 28 having opposed closed ends 30. The cylindrical container 28 defines an interior chamber 32 commensurate in length with the container 28, and a female protection item 34, preferably a feminine tampon, is held within the chamber 32 of the container 28. In addition, a feminine wipe 36 enclosed and sealed in a waterproof wrapping 38 is also placed in the container 28 and encircles the feminine item 34 so that the sanitary qualities of the feminine wipe 36 can be maintained and both the wipe 36 and the feminine tampon 34 can be easily stored within the chamber 32 of the container 28. A portion 40 of the body of the container 28 can be pulled back or peeled away to allow access to and removal of the contents held therein—which for the embodiment of FIGS. 3 and 4 is the feminine wipe 36 and the feminine tampon 34.

The feminine hygiene protection kit 26 of FIGS. 3 and 4 would be used in essentially the same manner as the feminine hygiene protection kit 10 of FIGS. 1, 2 and 5. Therefore, both embodiments 10 and 26 allow a woman to maintain a fresh and clean physical and mental state throughout an active and busy day while greatly alleviating the discomfiting inconveniences that accompanying the monthly menstrual cycle.

While the invention has been described with reference to specific embodiments thereof, it will be apparent to those skilled in the art that numerous modifications, alterations, and variations are possible and practicable, and accordingly, all such modifications, alterations, and variations are to be regarded as coming within the spirit and scope of the invention.

1. A feminine hygiene protection kit, comprising:
a lightweight, waterproof packaging including a cavity and an openable lid;
a feminine protection item contained within the packaging and for selective removal therefrom;
a feminine wipe contained within the packaging and removable concomitant with the removal of the feminine protection item; and
a wrapping enclosing the feminine wipe for maintaining the sanitary qualities of the feminine protection item and the feminine wipe.

2. The feminine hygiene protection kit of claim 1 wherein the feminine wipe is attached to the feminine protection item so that the wipe and the protection item can be removed together and simultaneous from the packaging.

3. The feminine hygiene protection kit of claim 2 wherein the packaging is square-shaped.

4. The feminine hygiene protection kit of claim 3 wherein the packaging is cylindrical-shaped.

5. A feminine hygiene protection kit for use by a woman for personal cleansing and freshening, comprising:
a lightweight, waterproof packaging having a chamber and a peel-off lid;
a feminine protection item storable within the chamber of the packaging and for removal therefrom for changing of the feminine protection item;
a feminine wipe storable within the chamber of the packaging and for removal therefrom for feminine cleansing and freshening during the process of changing from a used to a new feminine protection item;
a wrapping for enclosing the feminine wipe so that the sanitary qualities of the feminine wipe and the feminine protection item can be maintained; and
after using the feminine wipe the feminine wipe and the used feminine protection item are placed in the packaging so that the packaging can be sanitarily disposed of in a refuse container.

6. The feminine hygiene protection kit of claim 5 wherein the feminine wipe is attached to the feminine protection item so that the wipe can be removed from the packaging simultaneous with the feminine protection item.

7. The feminine hygiene protection kit of claim 6 wherein the packaging is square-shaped.

8. The feminine hygiene protection kit of claim 7 wherein the packaging is cylindrical-shaped.

9. The feminine hygiene protection kit of claim 8 wherein the feminine wipe is permeated with a distinct scent for enhancing the cleansing and freshening process.
